(19) United States
(12) Patent Application Publication
(10) Pub. No.: US 2008/0249425 A1
(43) Pub. Date: Oct. 9, 2008

(54) Method and Apparatus for Defining Cardiac Time Intervals

(75) Inventor: Robert Allan Phillips, New South Wales (AU)

Correspondence Address: MERCHANT & GOULD PC, P.O. BOX 2903, MINNEAPOLIS, MN, 55402-0903, US

(73) Assignee: USCOM LIMITED, Sydney (AU)

(21) Appl. No.: 11/661,623

(22) PCT Filed: Aug. 30, 2005
(86) PCT No.: PCT/AU2005/001313

§ 371 Date: Mar. 13, 2008

(30) Foreign Application Priority Data
Aug. 30, 2004 (AU) 2004904932

(51) Int. Cl. A61B 5/0468

(52) U.S. Cl. 600/516

(57) ABSTRACT

A method and apparatus for defining cardiac time intervals using the pre-systolic diastolic filling period and the proceeding systolic period. The diastolic period can begin at the end of the T wave and terminate at the onset of the proceeding Q wave as defined in electrocardiograph signals. The systolic period can begin at onset of the Q peak and terminate at the end of the T wave as defined in electrocardiograph signals.

### FIELD OF THE INVENTION

The present invention relates to the field of signal monitoring and, in particular, discloses a method of measuring time intervals from a cardiac signal.

### BACKGROUND OF THE INVENTION

Blood flow within the human body is cyclic and is responsive to the changing pressures generated by contraction and relaxation of the heart muscles. This cyclic contraction and relaxation produces pulsating blood flow. Known Doppler ultrasound techniques can measure the phase shift associated with this flow and display the signal on a screen. An example of such a system is provided in Patent Cooperation Treaty Publication Number WO 99/66835 entitled “Ultrasonic Cardiac Output Monitor”, the publication of which is incorporated herein by cross-reference.

The Doppler Ultrasound flow profiles provide important clinical information. An example of a time v. velocity flow profile is shown in FIG. 1 for a measurement taken utilising a machine constructed in accordance with techniques disclosed in the aforementioned PCT Publication.

Cardiac function can be described by measurement of time intervals relating to the systolic and diastolic phases of the heart beat as shown in the electrocardiograph signal of FIG. 2.

The conventional parameters in an electrocardiograph signal are defined as:
P wave: the sequential activation (depolarization) of the right and left atria;
QRS complex: right and left ventricular depolarization (normally the ventricles are activated simultaneously);
ST (T wave): ventricular repolarization;
PR interval: time interval from onset of atrial depolarization (P wave) to onset of ventricular depolarization (QRS complex),
QRS duration: duration of ventricular muscle depolarisation;
QT interval: duration of ventricular depolarization and repolarization.
RR interval: duration of ventricular cardiac cycle (an indicator of ventricular rate); and
PP interval: duration of atrial cycle (an indicator or atrial rate).

These intervals and their relationships are useful indices for measuring systolic and diastolic function. These intervals are described as being referenced from a full cardiac cycle beginning at the onset of ventricular activation, which has been conventionally defined as the onset of the electrocardiographic R wave 1.

Time intervals first became of interest in 1920, soon after the clinical introduction of the ECG in 1911. In a paper published in Heart (1920;7:353-70) Bazett published “An analysis of the time-relations of electrocardiograms” in which it was observed that systole represented a fixed 34.3% of cycle duration.

The concept of time intervals was further refined by Weissler who in 1977 published data in which the pre-ejection period (PEP), the time between onset of ventricular activation and the beginning of ejection time and ejection time (ET) were analysed (NEJM 1977;296:321-324. PEP was found to lengthen with disease, while ejection time shortened and PEP/LVET lengthened. Again the onset of electrocardiographic Q wave was determined to be the beginning of measurement of the time intervals.

The concept was expanded by Tei et al in 1995 (J Cardiol 1995;26:357-366) when the Tei Index, the sum of isovolumic contraction time and isovolumic relaxation time, divided by ejection time was proposed as a combined index of myocardial performance including both systolic and diastolic function. The basis for this measurement was that reduced systolic function was associated with increased IVCT and decreased ET, while diastolic dysfunction was associated with IVRT prolongation. This index has been proposed to evaluate many cardiac abnormalities and is a common component of echocardiographic assessment.

Time intervals in cardiac signals are conventionally measured from the systolic ejection and the proceeding diastolic period, with a manual trace beginning at the onset of systolic ejection and terminating at the completion of the proceeding diastole. While these time intervals have proven useful they are not universally representative of cardiovascular physiology with only moderate utilities and adoption.

### SUMMARY OF THE INVENTION

It is an object of the present invention to provide an improved method and system for cardiac measurement.

In accordance with a first aspect of the present invention, there is provided a method of defining cardiac time intervals using the pre-systolic diastolic filling period and the proceeding systolic period. The diastolic period can begin at the end of the T wave and terminate at the onset of the proceeding Q wave as defined in electrocardiograph signals. The systolic period can begin at onset of the Q peak and terminate at the end of the T wave as defined in electrocardiograph signals.

For echocardiographic applications the proposed measurement of time intervals will begin at the end of aortic valve closure, at the onset of isovolumic relaxation, the first component of diastole, and will continue to the onset of aortic valve opening, immediately post isovolumic contraction, the early phase of systole, and will continue to the end of systolic ejection or valve closure.

In accordance with a further aspect of the present invention, there is provided a method of monitoring the heart, the method including the step of utilising the pre-systolic diastolic filling period and the proceeding systolic period to derive an indicator of heart performance and thereby derive a new set of cardiac time interval analysis.

The method can also include the step of utilising the indicator in treatment of the heart.

In accordance with another aspect of the present invention, there is provided a method for measuring the cardiac period of a heart, the method comprising the steps of: (a) extracting a waveform indicative of cardiac activity within the heart; (b) determining a pre-systolic diastolic filling period and the proceeding systolic period; and (c) combining the pre-systolic diastolic filling period and the proceeding systolic period and outputting the combined result as a measure of cardiac period.

In accordance with a further aspect of the present invention, there is provide an apparatus for measuring the cardiac period of a heart, the apparatus comprising: a transducer for measuring physical or electrical activity associated with the heart; a processing unit for extracting a measure of a pre-systolic diastolic filling period and the proceeding systolic period; and a display for outputting the combination of the pre-systolic diastolic filling period and the proceeding systolic period as a measure of cardiac period.

### BRIEF DESCRIPTION OF THE DRAWINGS

Preferred embodiments of the present invention will now be described by way of example only with reference to the accompanying drawings in which:

FIG. 1 illustrates an example Doppler Flow Profile for measuring cardiac time intervals;

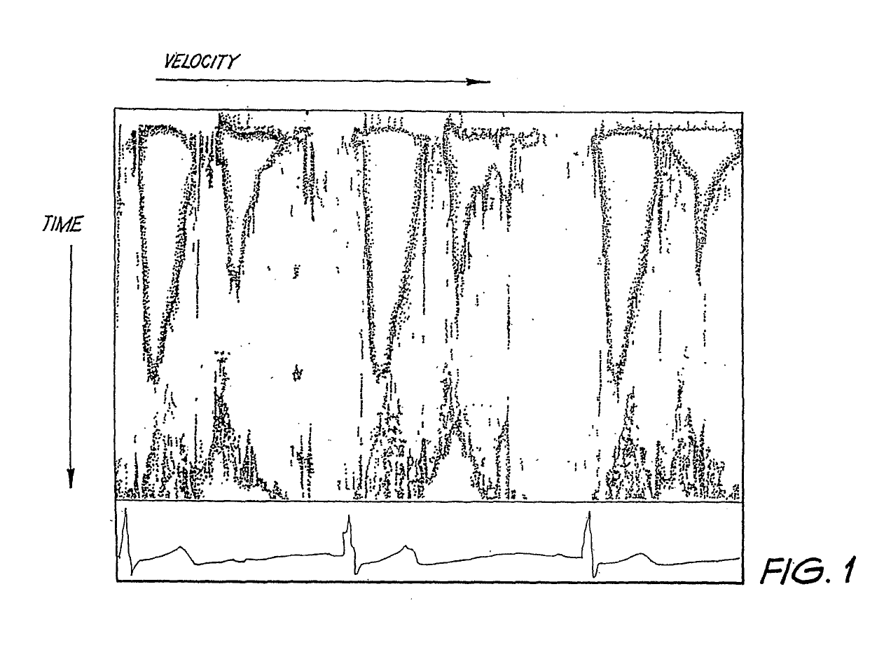

FIG. 2 illustrates the standard time intervals and peak designations of an electrocardiograph signal;

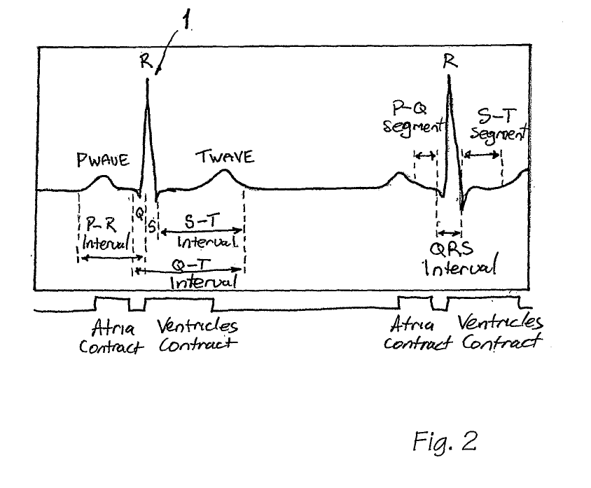

FIG. 3 illustrates a Doppler Flow Profile identifying cardiac time intervals demonstrating systolic ejection and preceding diastolic period;

FIG. 4 illustrates a screen dump of a Continuous Wave Doppler Device showing the Conventional Systolic Period and Presystolic period;

FIG. 5 illustrates an ECG trace identifying cardiac time intervals demonstrating systolic ejection and preceding diastolic period;

FIG. 6 illustrates the method steps of the preferred embodiment; and

FIG. 7 illustrates schematically one form of apparatus implemented to carry out the method of the preferred embodiment.

### DESCRIPTION OF THE PREFERRED AND OTHER EMBODIMENTS

The preferred embodiment provides a method for measuring cardiac time intervals which is more representative of cardiovascular physiology than commonly used practises and thereby leads to improved results. This leads to better assessment and management of the cardiac function.

Cardiac contraction and relaxation varies both in rate and force from beat to beat, so it has been found that a comprehensive understanding of the cardiac function is required for accurate analysis and diagnosis.

The Frank-Starling Law is one of the fundamental physiologic observations that describes cardiac performance. The Law relates ventricular filling with the proceeding cardiac systole. The cardiac output cycle ejects all the blood that returns to it during diastole without damming of blood in the cardiac veins. Intrinsic regulatory mechanisms permit adaptation of the heart to rates of venous return which may vary from about 2 litres per minute at rest to about 25 litres per minute during exercise.

Additionally, the Law relates the strength of the ventricular contraction, or systolic ejection, as being dependent on the degree of muscle stretch which is dependent on the left ventricular end diastolic volume (LVEDV), or the diastolic filling volume of the ventricle. The LVEDV is a critical determinant of left ventricular function.

During diastole, blood from the left atrium flows past the mitral valve to fill the left ventricle. The more the myocardium is stretched, the more the blood volume that is ejected during systole increases. The degree of myocardial stretch at the end of diastole is often referred to as preload; with increasing preload, up to a point, the work produced by the myocardium (left ventricular stroke work) increases. This relationship is the essence of Starling's law of the heart.

The degree of myocardial stretch is usually measured in vitro on isolated muscle preparations. In vivo, the degree of stretch correlates with the LVEDV. According to Starling's law, LVEDV is the left ventricular preload and bears the same relationship to left ventricular stroke work as does the degree of myocardial muscle stretch.

Stroke volume can also be substituted for left ventricular stroke work. Thus in vivo, Starling's law of the heart relates LVEDV to stroke volume. The higher the LVEDV (up to a point), the higher the stroke volume. Since cardiac output is the stroke volume times the heart rate, at a constant heart rate the same Starling relationship exists for LVEDV vs. cardiac output.

Intrinsic regulation depends on the fact that stretching cardiac muscle (during the diastolic period) results in a greater force of contraction (during the proceeding systolic period). Thus, increased venous return stretches the heart and causes increased force of contraction (and a moderate increase in heart rate), resulting in a corresponding increase in cardiac output.

The most accurate measurement of cardiac function in accordance with known physiological function of the heart is required to provide accurate diagnosis of cardiac related problems.

In the common physiologic measurement methods used to measure and monitor cardiac performance such as echocardiography, angiography and electrocardiography, there is a fundamental mismatch in the usual definition of cardiac time intervals and the physiology described by the Frank-Starling Law.

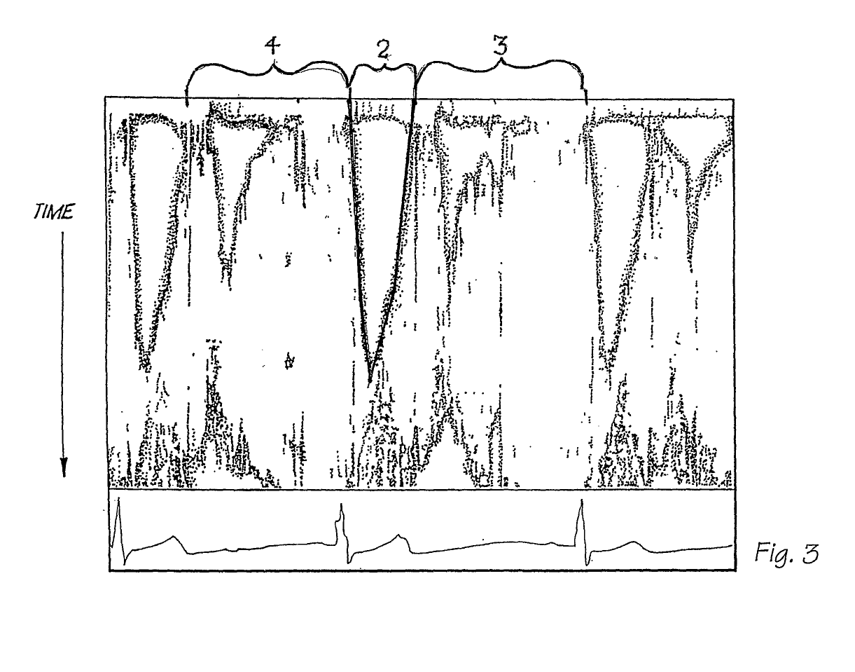

FIG. 3 shows a typical time v. velocity flow profile obtained using the Continuous Wave Doppler flow or echocardiographic (echo) method showing the systolic ejection period 2 and the proceeding diastolic period 3. The common method of defining the time intervals firstly considers the systolic period 2 and proceeding diastolic period 3 as secondary.

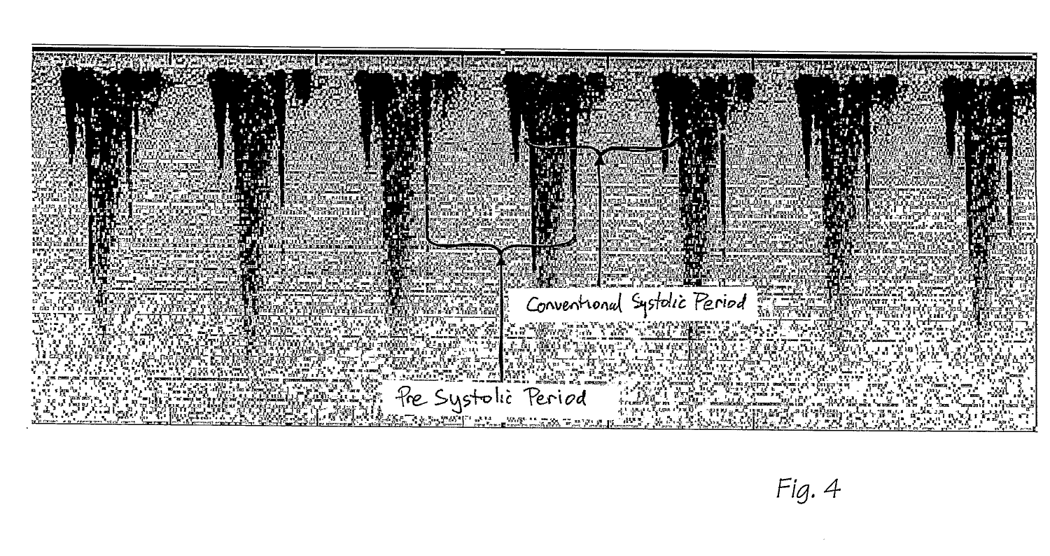

However, as shown in FIG. 3, it is the preceding diastolic period 4 which determines the cardiac output or stroke volume during the systolic period 2. FIG. 4 is a screen dump of a Doppler Flow image showing the relationship between the two periods.

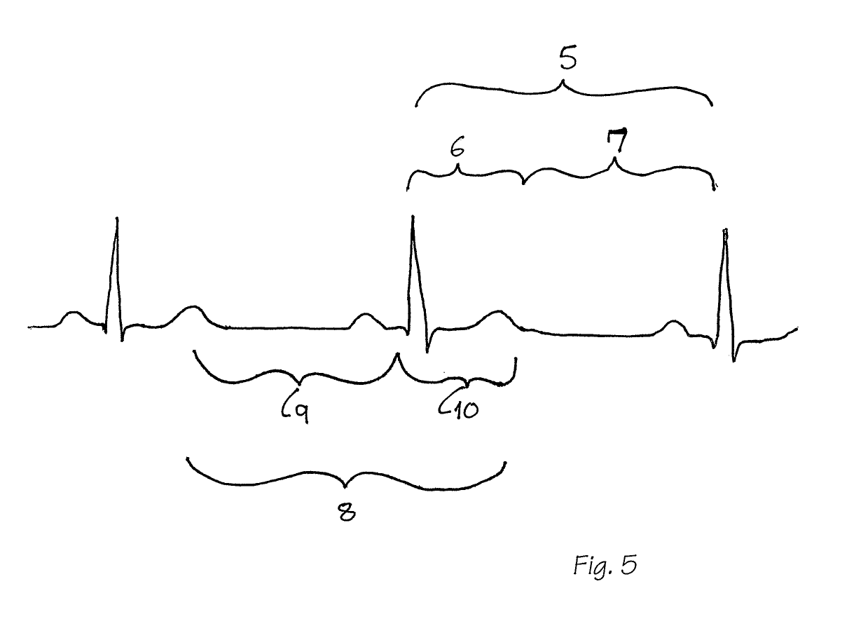

This method of determining cardiac time intervals using the pre-systolic diastolic period and the proceeding systolic period, has application to any method of measuring cardiac time intervals. For example, FIG. 5 shows a typical electrocardiograph signal with the conventional cycle 5 utilising the conventional systole 6 at the start of the cycle, and the conventionally used proceeding diastole 7. Using the current proposed method the proposed cycle 8 is used which in turn uses the pre-systolic diastole 9 and the proceeding systole 10.

The advantage of this method is that these new intervals more correctly represent cardiac physiology described by the Frank-Starling law, and provide improved accuracy, utility and adoption of cardiac time intervals. This potential improvement is particularly important in disease associated with heart rate variability (HRV) where the beat-to-beat fluctuations in the rhythm of the heart can provide an indirect measure of heart health.

Knowledge of the diastolic time periods and their direct affect on the subsequent systolic time periods is vital in ascertaining cardiac function. Particularly in the case of severe heart arrhythmia or in situations where detailed analysis and monitoring of heart function is needed, such as for example where a patient is under anaesthesia, substantial variations in subsequent heart beats can be rapidly detected and acted upon.

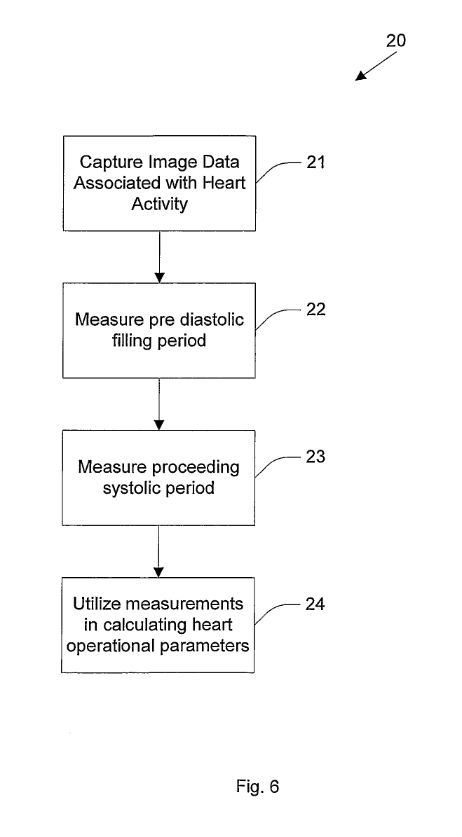

The method of the preferred embodiment can be readily implemented in monitoring devices via reprogramming of the equipment to monitor the cardiac time interval using the pre-diastolic filing period and the proceeding systolic period. The method can proceed in accordance with the steps 20 of FIG. 6 wherein image data, such as electrocardiograph or ultrasound measurements associated with heart activity are first captured. Next the pre-diastolic period is measured 22, followed by the proceeding systolic period 23. These measurements are then used to calculate near operational parameters 24.

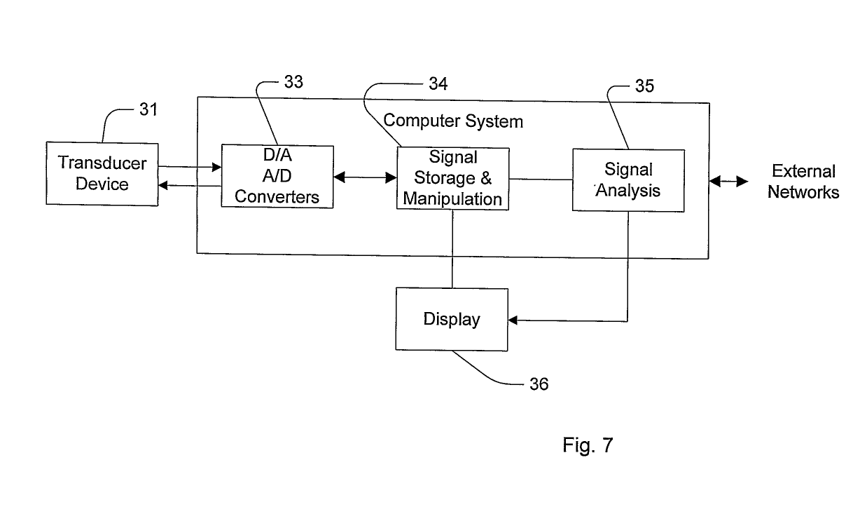

FIG. 7 illustrates schematically an arrangement for carrying out the preferred embodiment and includes a suitably reprogrammed system as described in the present inventor's PCT Patent Application Number PCT/AU99/00507 entitled “Ultrasonic Cardiac Output Monitor” the contents of which are hereby incorporated by cross reference. In the system arrangement 30, a transducer device 31 is operated by a series of A/D and D/A converts 33 so as to emit and receive the Continuous Wave Doppler ultrasound signal. This signal is fed to a signal storage and manipulation unit which can comprise a high end Digital Signal Processor device 34, microcontroller and frame and program memory storage. Here the signal is readied for display on Display 36. Additionally, the signal is subjected to digital image processing and analysis so as to determine the cardiac period. This can proceed by many different techniques outlined in the textbooks such as “Digital Image Processing” by Gonzalez & Woods. Importantly, the pre diastolic filing period and the proceeding systolic period are used to measure the cardiac period. Upon measurement, the output can be numerically displayed on display 36.

The preferred embodiment includes utilising this measurement in deriving other measures utilised in the treatment of the heart. This includes providing monitoring values associated with the measurement.

The forgoing describes only one embodiment of the present invention. Modifications, obvious to those skilled in the art, can be made thereto without departing from the scope of the invention.

THE CLAIMS DEFINING THE INVENTION ARE AS FOLLOWS:

1. A method of performing heart monitoring, the method including the step of defining cardiac time intervals using the pre-systolic diastolic filling period and the proceeding systolic period.

2. A method as claimed in claim 1 wherein said diastolic period begins at the T wave and terminates at onset of the proceeding QRS, as defined in electrocardiograph signals.

3. A method as claimed in claim 1 wherein said systolic period begins at the onset of the Q wave and terminates at the T wave peak, as defined in electrocardiograph signals.

4. A method of monitoring the heart, the method including the step of utilising the pre-systolic diastolic filling period and the proceeding systolic period to derive an indicator of heart performance.

5. A method as claimed in claim 4 further comprising the step of utilising said indicator in treatment of the heart.

6. A method for measuring the cardiac period of a heart, the method comprising the steps of:
(a) extracting a waveform indicative of cardiac activity within the heart;
(b) determining a pre-systolic diastolic filling period and the proceeding systolic period; and
(c) combining the pre-systolic diastolic filling period and the proceeding systolic period and outputting the combined result as a measure of cardiac period.

7. An apparatus for measuring the cardiac period of a heart, the apparatus comprising:
a transducer for measuring physical or electrical activity associated with the heart;
processing unit for extracting a measure of a pre-systolic diastolic filling period and the proceeding systolic period;
display for outputting the combination of the pre-systolic diastolic filling period and the proceeding systolic period as a measure of cardiac period.

8. An apparatus as claimed in claim 7 wherein said transducer is a continuous wave Doppler transducer.
